(19) United States
(12) Patent Application Publication
(10) Pub. No.: US 2015/0151097 A1
(43) Pub. Date: Jun. 4, 2015

(54) NANOFLUIDIC DELIVERY SYSTEM

(71) Applicant: Biltmore Technologies, Inc., Palm Beach, FL (US)

(72) Inventors: David Carnahan, Needham, MA (US); Nolan Nicholas, Worcester, MA (US); Kyle G. Fohrman, Palm City, FL (US); Howard Busch, Lantana, FL (US); Thomas T. Morgan, Palm Beach, FL (US); Troy G. Fohrman, Palm City, FL (US)

(21) Appl. No.: 14/558,503

(22) Filed: Dec. 2, 2014

Related U.S. Application Data
(60) Provisional application No. 61/910,486, filed on Dec. 2, 2013.
(60) Provisional application No. 61/910,491, filed on Dec. 2, 2013.

(51) Int. Cl. A61M 37/00; B21G 1/08

(52) U.S. Cl. CPC A61M 37/0015; B21G 1/08; A61M 2037/0023; A61M 2037/0053; A61M 2037/0061

(57) ABSTRACT

Apparatus for subcutaneously delivering a substance to a patient, said apparatus comprising:
a carrier comprising a flexible body, wherein said flexible body comprises a reservoir, and further wherein said reservoir contains the substance which is to be delivered to the patient;
a nanoneedle assembly comprising:
   a tubular body having a distal end and a proximal end;
   a base plate movably mounted intermediate said distal end and said proximal end of said tubular body, said base plate comprising a distal surface and a proximal surface, with a plurality of through-holes extending between said distal surface and said proximal surface of said base plate, said proximal surface of said base plate being in fluid communication with said reservoir;
   a plurality of nanoneedles, wherein each of said plurality of nanoneedles comprises a distal end, a proximal end, and a lumen extending therebetween, said proximal end of each of said plurality of nanoneedles being mounted to said base plate such that said lumen of each of said plurality of nanoneedles is in fluid communication with said through-holes of said base plate;
   a fixed guide plate mounted at said distal end of said tubular body, said fixed guide plate comprising a plurality of through-holes extending therethrough, said through-holes of said fixed guide plate being sized to receive said distal ends of said plurality of nanoneedles; and
   a moveable guide plate disposed intermediate said base plate and said fixed guide plate, said moveable guide plate comprising a plurality of through-holes extending therethrough, said through-holes of said movable guide plate being sized to receive said plurality of nanoneedles, such that said plurality of nanoneedles extend through said through-holes of said movable guide plate; and
   at least one spring tab for biasing said movable guide plate away from said fixed guide plate;
wherein, when said base plate is moved distally, said movable guide plate moves distally, such that said movable guide plate provides lateral support to said nanoneedles, whereby to prevent buckling of said nanoneedles; and
wherein when said base plate moves distally, said distal end of each of said plurality of nanoneedles passes through said through-holes of said fixed guide plate into the patient, and further wherein when said distal ends of said plurality of nanoneedles are disposed distally of said fixed guide plate, the substance within said reservoir passes through each of said lumens of said plurality of nanoneedles, whereby to deliver the substance to the patient.

### REFERENCE TO PENDING PRIOR PATENT APPLICATION

This patent application:

(i) claims benefit of pending prior U.S. Provisional Patent Application Ser. No. 61/910,486, filed Dec. 2, 2013 by Paradox Private Equity Funds, LLC and Troy G. Fohrman et al. for NANOFLUIDIC DELIVERY SYSTEM (Attorney's Docket No. FOHRMAN-1 PROV); and

(ii) claims benefit of pending prior U.S. Provisional Patent Application Ser. No. 61/910,491, filed Dec. 2, 2013 by Paradox Private Equity Funds, LLC and David Carnahan et al. for NANOFLUIDIC DELIVERY SYSTEM (Attorney's Docket No. FOHRMAN-2 PROV).

The two (2) above-identified patent applications are hereby incorporated herein by reference.

### FIELD OF THE INVENTION

This invention relates to medical apparatus and procedures in general, and more particularly to needles for the subcutaneous delivery of a substance to a patient.

### BACKGROUND OF THE INVENTION

In many situations, a substance (e.g., a biologically-active material such as a pharmaceutical, nutriceuticals, hormone, medical food, chemical agent, etc., or a biologically-inert material such as a reconstructive agent, or GRAS (“Generally Recognized As Safe”) molecule(s), etc.) may need to be administered to the patient. In some cases, substances may be delivered through multiple areas including, but not limited to: oral, nasal, rectal, ocular and cutaneous sites. However, in some cases, the substance may need to be delivered by subcutaneous or intravenous injection rather than by a transdermal vehicle.

It is well known that using a conventional needle for intramuscular or intravenous injection causes discomfort (i.e., pain) for the patient. Moreover, because conventional needles cause discomfort for a patient, the patient may be apprehensive and seek to avoid this form of administration, even when medically necessary, which will ultimately affect the ability of the clinician to adequately treat the patient. Additionally, many of the newer medications are protein-based macromolecules, complex sugars, fusion proteins and monoclonal antibodies. These macromolecules are not deliverable without the use of traditional intravenous (IV), subcutaneous (SQ), or intramuscular (IM) needles, so patients are currently forced to undergo the discomfort and apprehension associated with conventional needles.

There are also, currently, limitations with respect to the effective delivery of GRAS substances in vivo for cosmetic preparations. Some recent delivery systems utilizing solid, non-hollow, microneedles have been devised whereby a coating of the GRAS substance is disposed on the outer diameter of the microneedle and then, using a method of movement, such as a roller, the GRAS substance is “pushed” into the surface of the skin. An alternative approach has been to lather a layer of GRAS-substance-containing lotion or cream on the skin's surface and then use the solid microneedles to “push” the substance into the skin. However, the delivery of GRAS substances by either method involving solid microneedles has not been painless.

Thus, there is a need for a new and improved means for painless delivery of substances (e.g., a biologically-active material such as a pharmaceutical, a hormone, a chemical agent, etc., or a biologically-inert material such as a reconstructive agent, GRAS molecule(s), etc.) through the skin of a patient by a needle.

### SUMMARY OF THE INVENTION

The present invention provides a new and improved means for painlessly delivering a substance (e.g., a biologically-active material such as a pharmaceutical, hormone, medical food, chemical agent, etc., or a biologically-inert material such as a reconstructive agent, GRAS molecule(s), etc.) through the skin of a patient by a needle.

More particularly, the present invention comprises the provision and use of a nanofluidic delivery system which comprises an array of nanoneedles for painless delivery of a substance transcutaneously to the patient. Significantly, the nanoneedles are sufficiently small as to permit painless penetration through the skin of the patient, so as to provide a pain-free injection to the patient.

In one preferred form of the invention, there is provided apparatus for subcutaneously delivering a substance to a patient, said apparatus comprising:
a carrier comprising a flexible body, wherein said flexible body comprises a reservoir, and further wherein said reservoir contains the substance which is to be delivered to the patient;
a nanoneedle assembly comprising:
   a tubular body having a distal end and a proximal end;
   a base plate movably mounted intermediate said distal end and said proximal end of said tubular body, said base plate comprising a distal surface and a proximal surface, with a plurality of through-holes extending between said distal surface and said proximal surface of said base plate, said proximal surface of said base plate being in fluid communication with said reservoir;
   a plurality of nanoneedles, wherein each of said plurality of nanoneedles comprises a distal end, a proximal end, and a lumen extending therebetween, said proximal end of each of said plurality of nanoneedles being mounted to said base plate such that said lumen of each of said plurality of nanoneedles is in fluid communication with said through-holes of said base plate;
   a fixed guide plate mounted at said distal end of said tubular body, said fixed guide plate comprising a plurality of through-holes extending therethrough, said through-holes of said fixed guide plate being sized to receive said distal ends of said plurality of nanoneedles; and
   a moveable guide plate disposed intermediate said base plate and said fixed guide plate, said moveable guide plate comprising a plurality of through-holes extending therethrough, said through-holes of said movable guide plate being sized to receive said plurality of nanoneedles, such that said plurality of nanoneedles extend through said through-holes of said movable guide plate; and
   at least one spring tab for biasing said movable guide plate away from said fixed guide plate;
wherein, when said base plate is moved distally, said movable guide plate moves distally, such that said movable guide plate provides lateral support to said nanoneedles, whereby to prevent buckling of said nanoneedles; and
wherein when said base plate moves distally, said distal end of each of said plurality of nanoneedles passes through said through-holes of said fixed guide plate into the patient, and further wherein when said distal ends of said plurality of nanoneedles are disposed distally of said fixed guide plate, the substance within said reservoir passes through each of said lumens of said plurality of nanoneedles, whereby to deliver the substance to the patient.

In another preferred form of the invention, there is provided a method for subcutaneously delivering a substance to a patient, said method comprising:
providing apparatus comprising:
   a carrier comprising a flexible body, wherein said flexible body comprises a reservoir, and further wherein said reservoir contains the substance which is to be delivered to the patient;
   a nanoneedle assembly comprising:
      a tubular body having a distal end and a proximal end;
      a base plate movably mounted intermediate said distal end and said proximal end of said tubular body, said base plate comprising a distal surface and a proximal surface, with a plurality of through-holes extending between said distal surface and said proximal surface of said base plate, said proximal surface of said base plate being in fluid communication with said reservoir;
      a plurality of nanoneedles, wherein each of said plurality of nanoneedles comprises a distal end, a proximal end, and a lumen extending therebetween, said proximal end of each of said plurality of nanoneedles being mounted to said base plate such that said lumen of each of said plurality of nanoneedles is in fluid communication with said through-holes of said base plate;
      a fixed guide plate mounted at said distal end of said tubular body, said fixed guide plate comprising a plurality of through-holes extending therethrough, said through-holes of said fixed guide plate being sized to receive said distal ends of said plurality of nanoneedles; and
      a moveable guide plate disposed intermediate said base plate and said fixed guide plate, said moveable guide plate comprising a plurality of through-holes extending therethrough, said through-holes of said movable guide plate being sized to receive said plurality of nanoneedles, such that said plurality of nanoneedles extend through said through-holes of said movable guide plate; and
      at least one spring tab for biasing said movable guide plate away from said fixed guide plate;
   wherein, when said base plate is moved distally, said movable guide plate moves distally, such that said movable guide plate provides lateral support to said nanoneedles, whereby to prevent buckling of said nanoneedles; and
   wherein when said base plate moves distally, said distal end of each of said plurality of nanoneedles passes through said through-holes of said fixed guide plate into the patient, and further wherein when said distal ends of said plurality of nanoneedles are disposed distally of said fixed guide plate, the substance within said reservoir passes through each of said lumens of said plurality of nanoneedles, whereby to deliver the substance to the patient;
positioning said apparatus such that said distal end of said tubular body is disposed against the skin of the patient;
moving said base plate distally so as to advance said plurality of nanoneedles into the skin of the patient; and
delivering the substance through said nanoneedles into the patient.

In another preferred form of the invention, there is provided a method for forming a hollow tube, said method comprising:
providing a support plate having a plurality of holes extending therethrough;
inserting a plurality of fibers into said plurality of holes so as to mount said fibers to said support plate;
overcoating said fibers with a stiff material;
removing said stiff material from the ends of said fibers opposite said support plate, whereby to expose said fibers; and
selectively etching away said fibers so as to leave hollow tubes of said stiff material extending from said support plate.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts and further wherein:

FIGS. 1-4 are schematic views showing a novel nanofluidic delivery system formed in accordance with the present invention;

FIG. 5 is a schematic view showing the nanoneedle assembly of the novel nanofluidic delivery system of FIGS. 1-4, with the fixed guide plate removed for clarity;

FIG. 6 is a schematic view showing the movable base plate and nanoneedles of the nanoneedle assembly of FIG. 5;

FIGS. 7-11 are schematic views showing further details of the novel nanofluidic delivery system of FIGS. 1-4 (note that in FIGS. 7, 9, 10 and 11, the bottom surface of flexible body 20 and the bottom surface of nanoneedle assembly 15 are shown slightly offset from one another for the purposes of better illustrating the bottom surface of nanoneedle assembly 15);

FIGS. 12 and 13 are exploded views of the nanofluidic delivery system of FIGS. 1-4 and 7-11;

FIG. 14 is a schematic view of the nanoneedle assembly of the nanofluidic delivery system of FIGS. 1-4 and 7-11;

FIGS. 15-17 are schematic views showing how nanoneedles will buckle when they are not properly supported intermediate their length;

FIGS. 18-22 are schematic views showing how the nanoneedles may be formed by carbon nanotubes (CNTs);

FIGS. 23A-23E, 24 and 25 are schematic views showing how a plurality of nanofibers may be arranged to form a hollow tubular meta-structure;

FIGS. 26A-26E are schematic views showing how nanoneedles may be formed by sacrificial fibers overplated with a rigid material; and

FIGS. 27 and 28 show an exemplary tungsten tubular structure, formed in accordance with the process depicted in FIGS. 26A-26E, extending out of the skin of a patient.

### DETAILED DESCRIPTION OF PREFERRED EMBODIMENTS

The present invention provides a new and improved means for painlessly delivering a substance (e.g., a biologically-active material such as a pharmaceutical, a hormone, a chemical agent, etc., or a biologically-inert material such as a reconstructive agent, etc.) through the skin of a patient by a needle.

More particularly, the present invention comprises the provision and use of a nanofluidic delivery system which comprises an array of nanoneedles for painlessly delivering a substance through the skin of a patient. Significantly, the nanoneedles are sufficiently small as to permit painless penetration through the skin of the patient, whereby to provide pain-free injection of a substance into the patient.

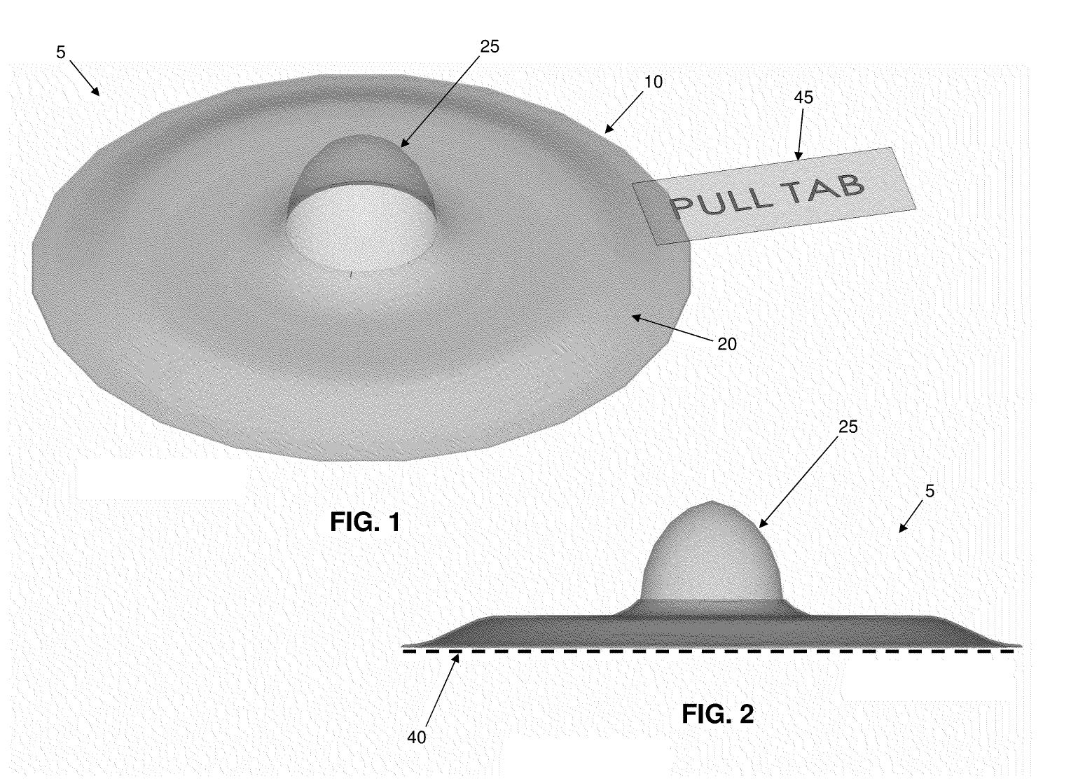

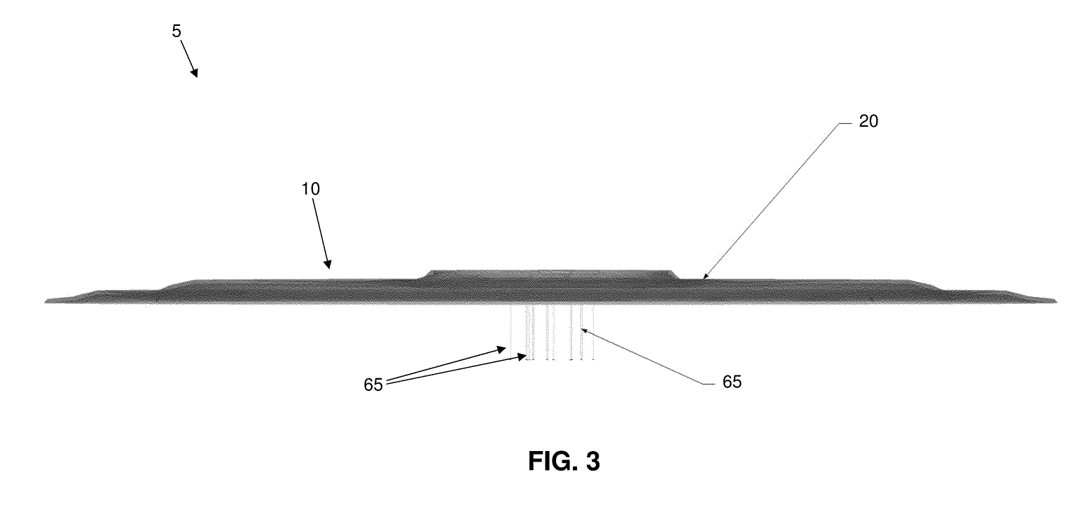

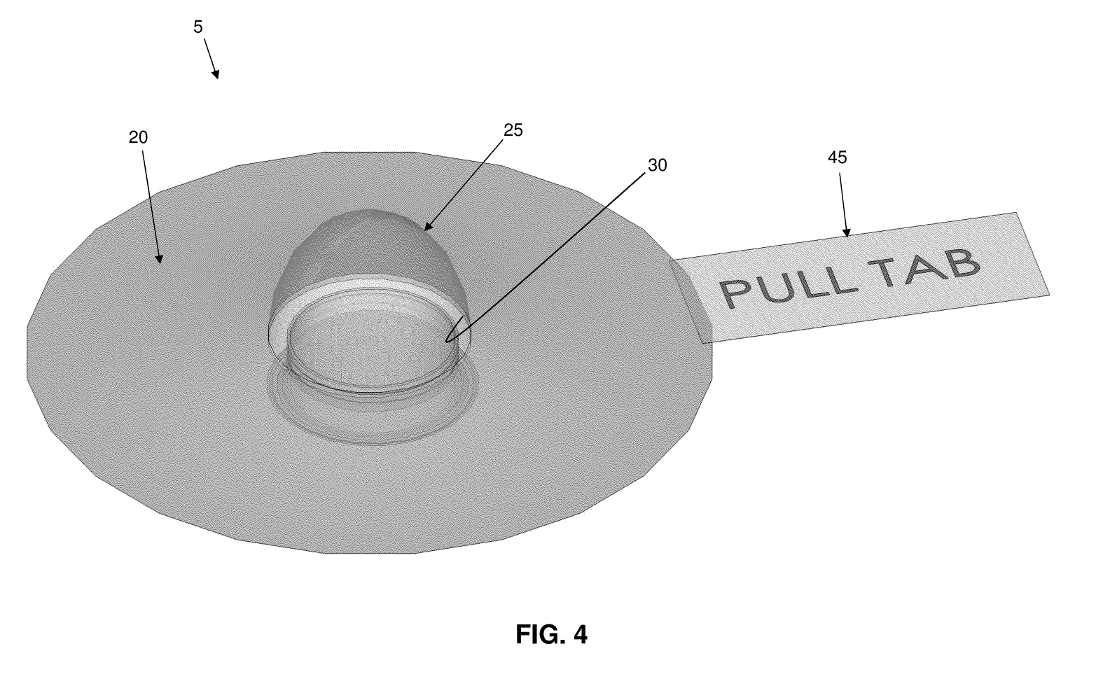

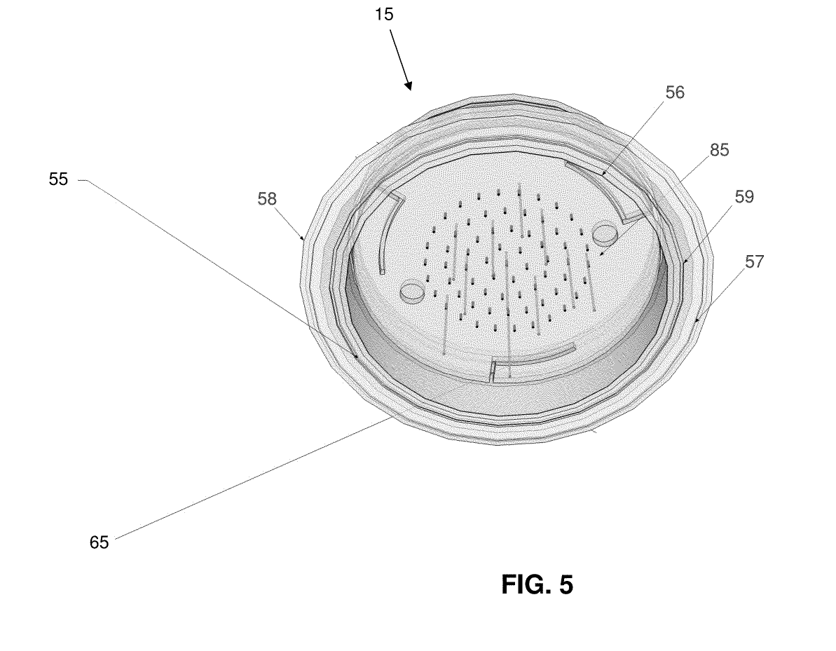

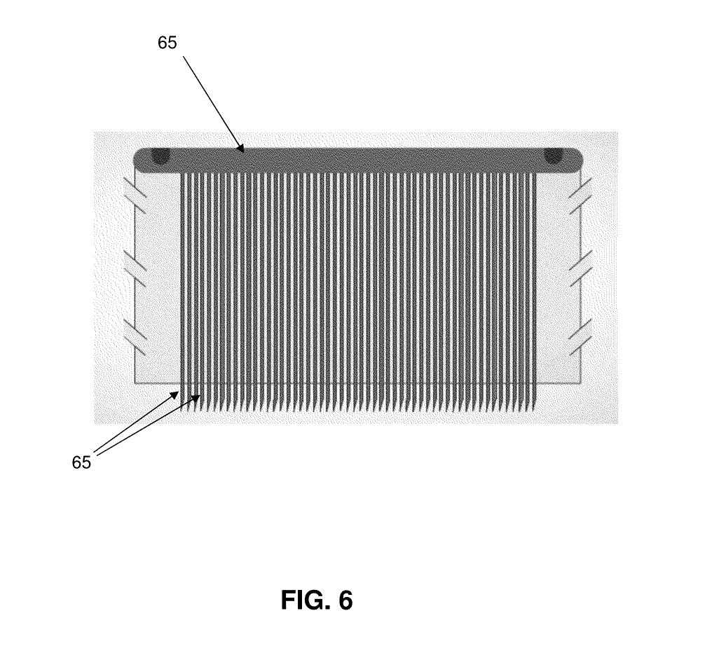

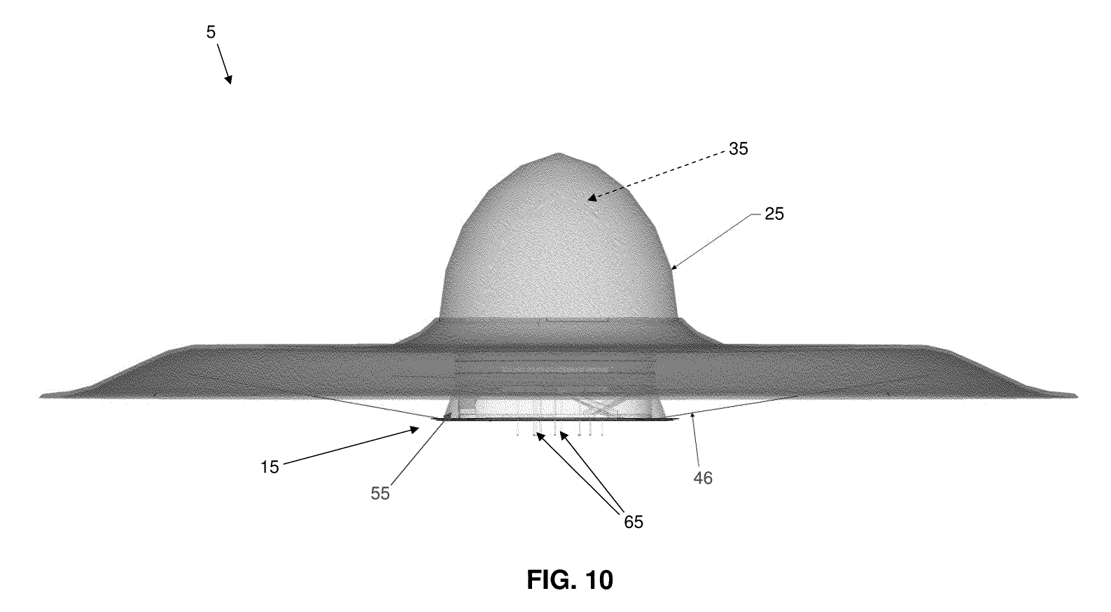

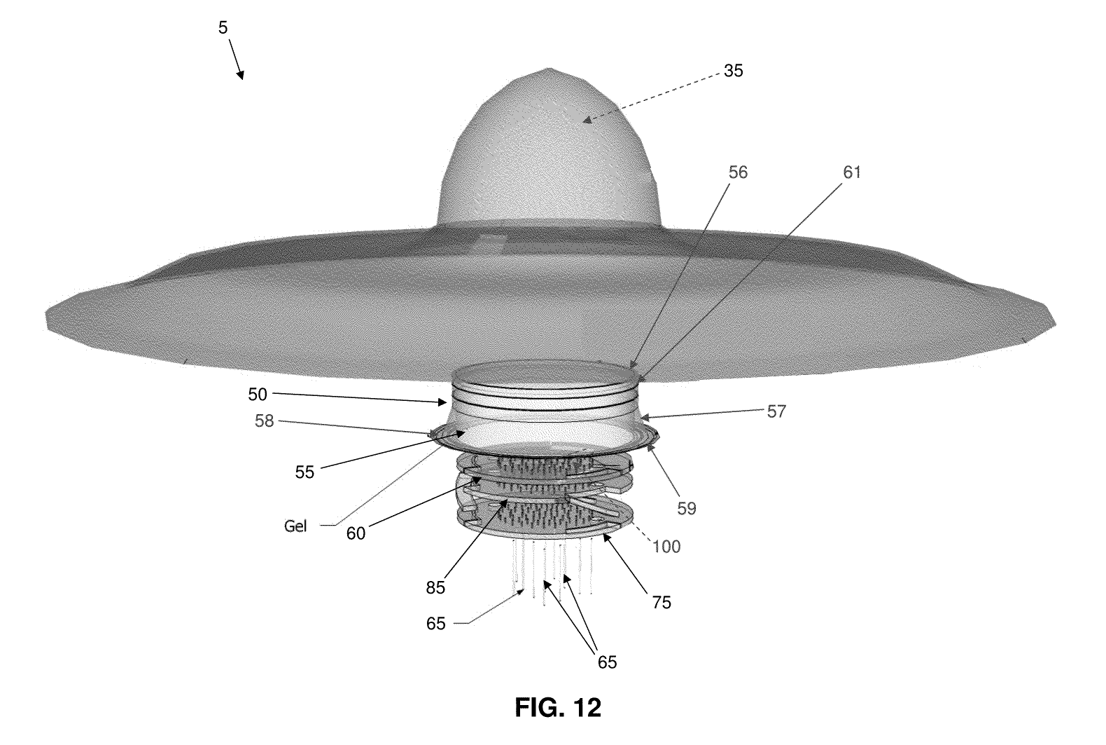

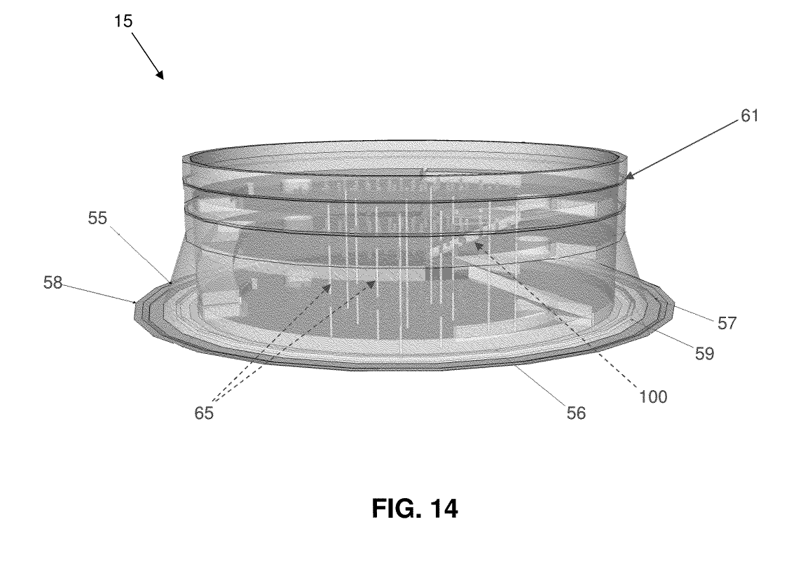

In one form of the present invention, and looking first at FIGS. 1-14, there is provided a nanofluidic delivery system 5 which generally comprises a carrier 10 and a nanoneedle assembly 15.

Carrier 10 generally comprises a flexible body 20 having a flexible dome 25 formed therein. Dome 25 has a concavity 30 formed therein. Nanoneedle assembly 15 is mounted across the base of concavity 30 so that nanoneedle assembly 15 and concavity 30 together define a reservoir 35 disposed within dome 25 and above nanoneedle assembly 15. Reservoir 35 contains the substance which is to be injected into the patient (e.g., a biologically-active material such as a pharmaceutical, a hormone, a chemical agent, etc., or a biologically-inert material such as a reconstructive agent, etc.). Preferably, a peel-away strip 40 covers the bottom surface of flexible body 20, sealing nanoneedle assembly 15. A pull tab 45 allows peel-away strip 40 to be removed at the time of use.

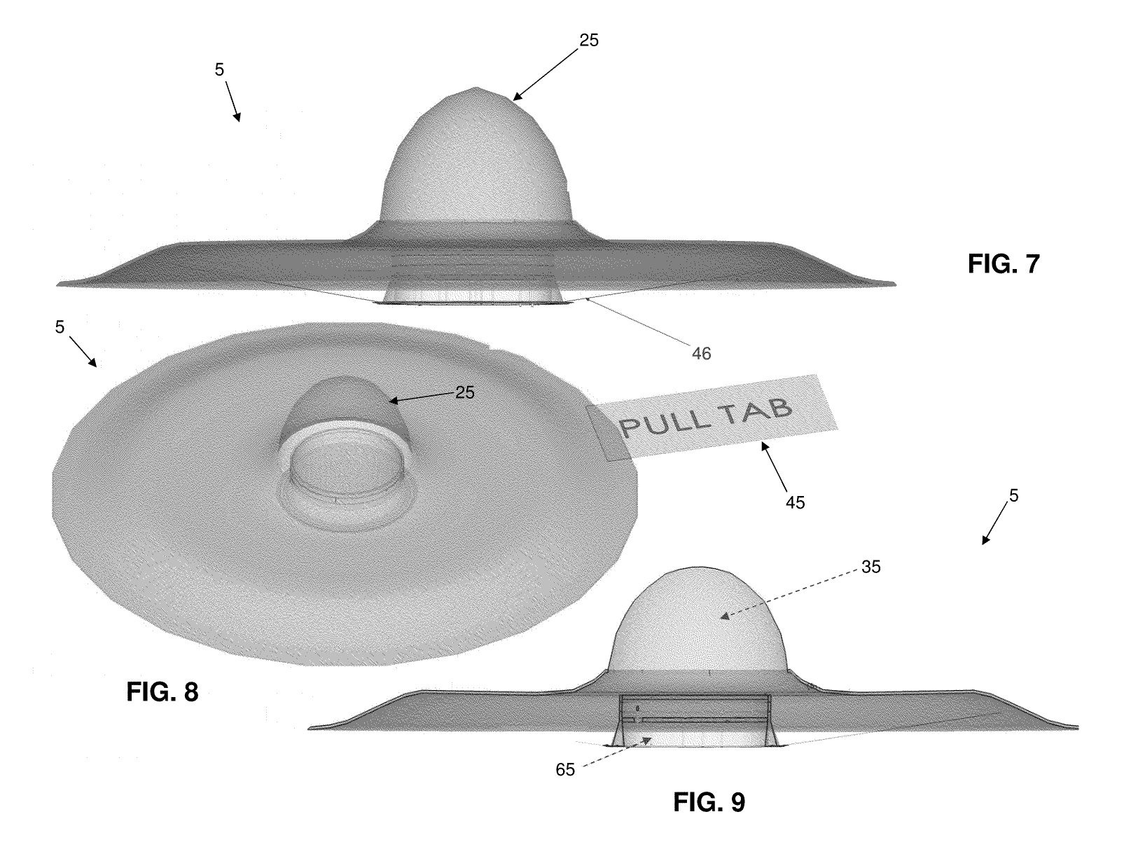

Nanoneedle assembly 15 comprises a tubular body 50 which is secured to flexible body 20 so that tubular body 50 communicates with reservoir 35 in dome 25. By way of example but not limitation, nanoneedle assembly 15 may also be secured to flexible body 20 via a lower support membrane 46 extending between flexible body 20 and the distal end of nanoneedle assembly 15 (see FIGS. 7 and 9-11).

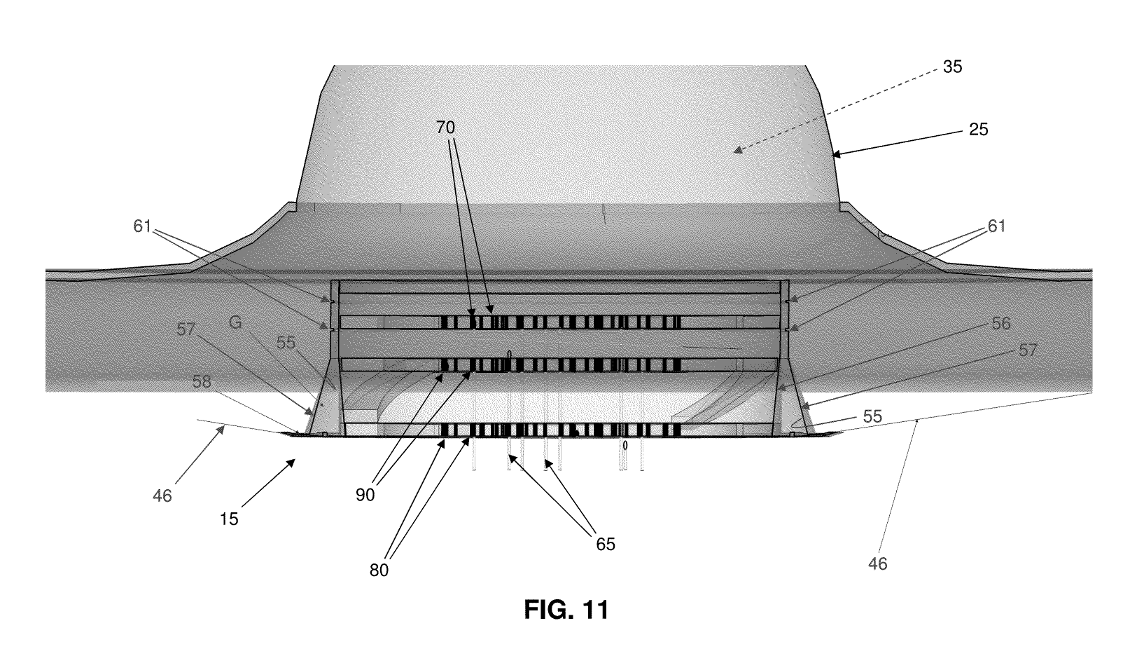

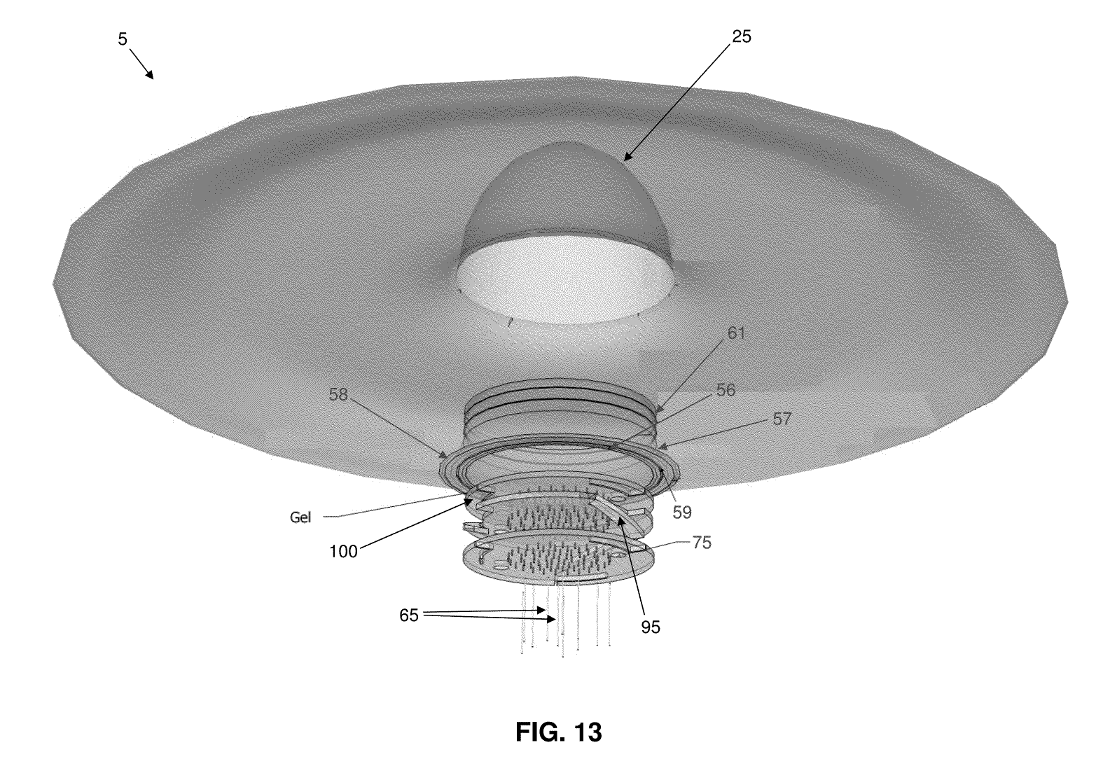

In one preferred form of the invention, and looking now at FIGS. 11 and 13, tubular body 50 comprises a gel reservoir 55 at the distal end of tubular body 50, such that gel G within gel reservoir 55 can contact the skin of the patient when peel-away strip 40 has been removed and nanofluidic delivery system 5 has been placed against the skin of a patient. More particularly, with this form of the invention, tubular body 50 comprises an outer wall 56. A gel reservoir wall 57 is disposed circumferentially around outer wall 56. A membrane cuff 58 is disposed circumferentially around the distal end of tubular body 50 and extends radially outboard from outer wall 56 such that the distal end of gel reservoir wall 57 contacts membrane cuff 58, thereby defining gel reservoir 55 as the volume bounded by outer wall 56, gel reservoir wall 57 and membrane cuff 58. If desired, an annular slit 59 (FIG. 13) may be formed in membrane cuff 58, so as to allow for the release of gel G from gel reservoir 55. A plurality of vents 61 may be formed in gel reservoir wall 57 so as to allow air to enter gel reservoir 55, thereby facilitating movement of gel G out of gel reservoir 55 through slit 59.

A movable base plate 60 is movably mounted within tubular body 50. Movable base plate 60 has an array of hollow nanoneedles 65 extending therefrom. More particularly, movable base plate 60 comprises a plurality of through-holes 70. Each through-hole 70 has a nanoneedle 65 extending therefrom, so that the lumen of the nanoneedle communicates with the region above movable base plate 60, i.e., with reservoir 35 in dome 25. Nanoneedles 65 are sufficient in number to deliver the desired quantity of a substance from reservoir 35 to the tissue of the patient within the desired time.

Each nanoneedle 65 is sized so as to be (i) long enough to penetrate the skin of a patient, and (ii) narrow enough to avoid causing pain to the patient. By way of example but not limitation, each nanoneedle 65 is preferably at least about 5 mm long and is preferably less than about 50 microns in diameter, and preferably has an interior lumen of at least about 10 microns.

Nanoneedles 65, which are at least about 5 mm long and less than about 50 microns in diameter, and preferably have an interior lumen of at least about 10 microns, tend to “buckle” easily, due to their extremely small size, their height-to-width aspect ratio, and the column strength attainable with current materials. To this end, nanoneedle assembly 15 provides lateral support for nanoneedles 65, both when they are contained within nanoneedle assembly 15 and when they are projected out of nanoneedle assembly 15 and into the skin of a patient.

More particularly, a fixed guide plate 75 is disposed at the distal end of tubular body 50. Fixed guide plate 75 comprises a plurality of through-holes 80. Each nanoneedle 65 extends through a through-hole 80 in fixed guide plate 75, whereby to provide lateral support for each nanoneedle 65 as the nanoneedle sits within nanoneedle assembly 15 and as the nanoneedle advances out of nanoneedle assembly 15 and into the skin of a patient.

In addition, a movable guide plate 85 is disposed intermediate movable base plate 60 and fixed guide plate 75. Movable guide plate 85 comprises a plurality of through-holes 90. Each nanoneedle 65 extends through a through-hole 90 in movable guide plate 85, whereby to provide lateral support for each nanoneedle 65 as the nanoneedle sits within nanoneedle assembly 15 and as the nanoneedle advances out of nanoneedle assembly 15 and into the skin of a patient.

Significantly, movable guide plate 85 comprises spring tabs 95 which spring-bias movable guide plate 85 away from fixed guide plate 75. Spring tabs 95 help ensure that movable guide plate 85 initially sits intermediate fixed guide plate 75 and movable base plate 60. At the same time, spring tabs 95 allow movable guide plate 85 to remain disposed intermediate movable base plate 60 and fixed guide plate 75 when movable guide plate 85 is advanced distally with movable base plate 60 during advancement of nanoneedles 65, whereby to provide lateral support for the nanoneedles during insertion into the skin of a patient. If desired, spring tabs 95 may be formed from a portion of movable guide plate 85.

Additionally, movable base plate 60 may also comprise spring tabs 100 which spring-bias movable base plate 60 away from movable guide plate 85. Spring tabs 100 help ensure that movable base plate 60 initially sits at the proximal end of tubular body 50, separated from movable base plate 60. At the same time, spring tabs 100 allow movable base plate 60 to advance distally within tubular body 50, whereby to allow advancement of nanoneedles 65 during insertion into the skin of a patient. If desired, spring tabs 100 may be formed from a portion of movable base plate 60.

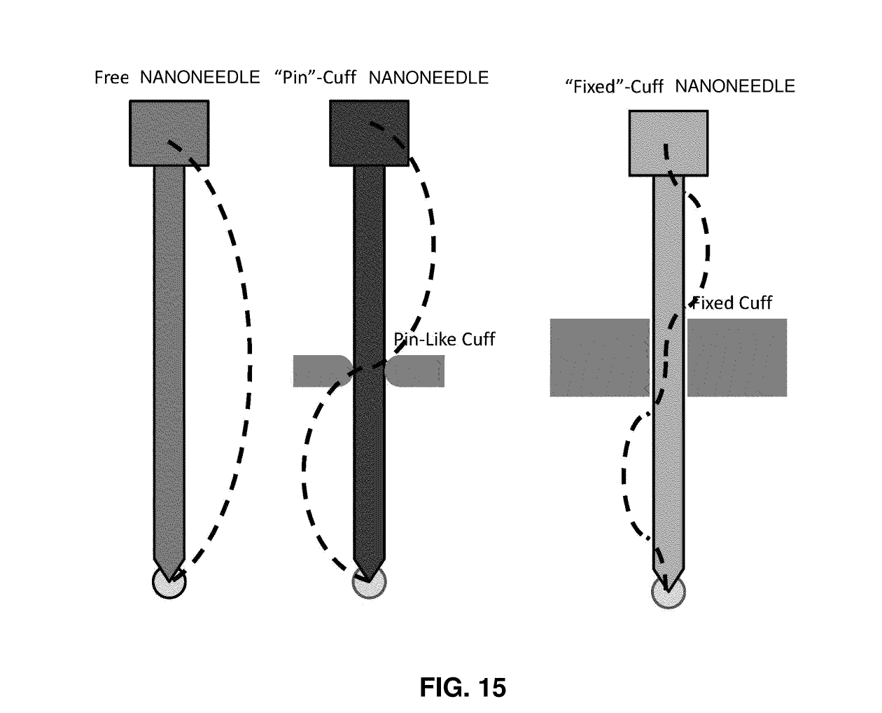

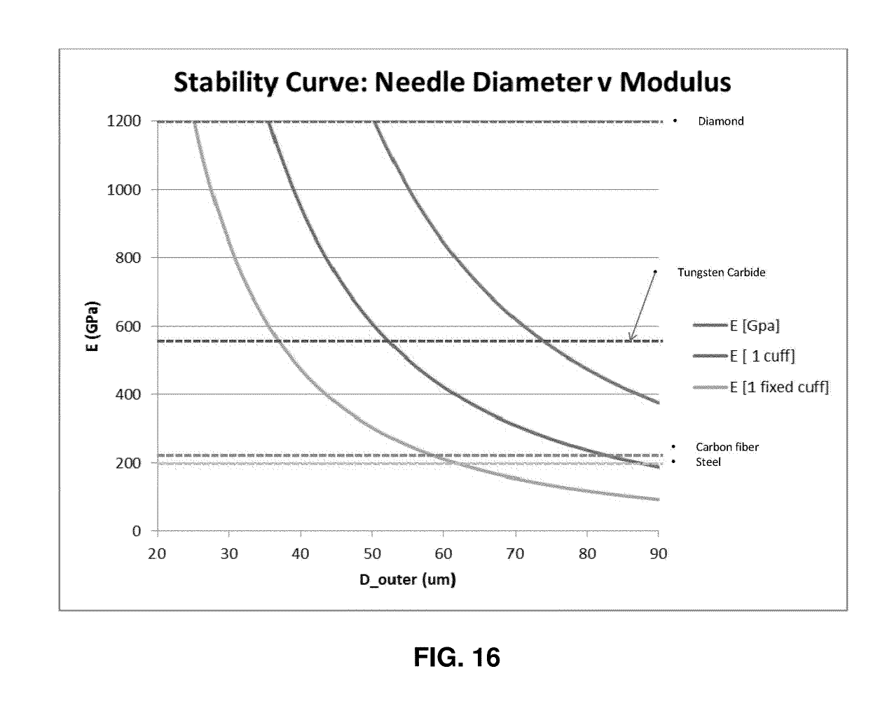

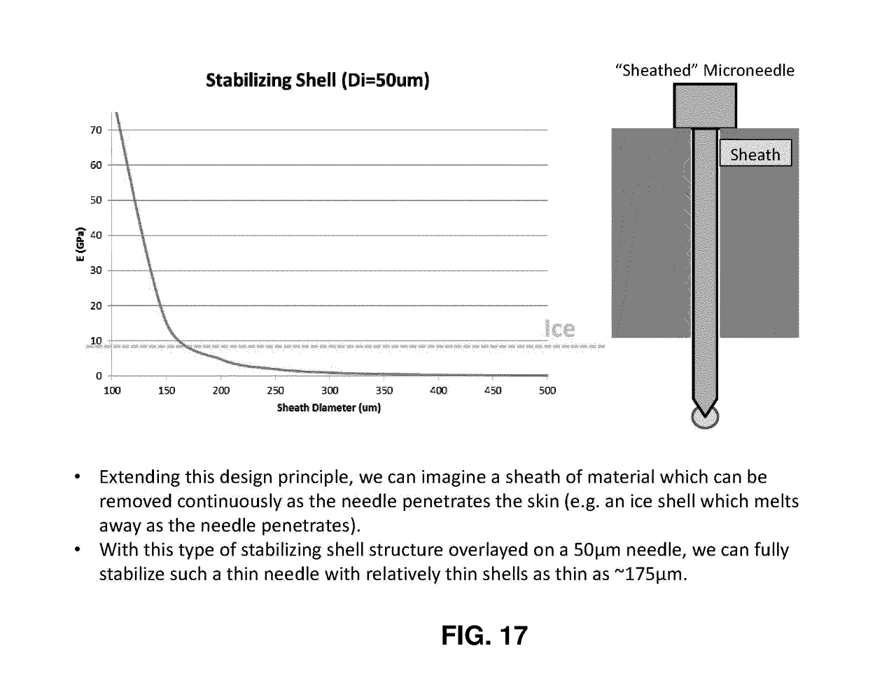

The provision of the movable guide plate 85 intermediate fixed guide plate 75 and movable base plate 60 is a significant feature, since it allows moving support for nanoneedles 65 during their advancement into the patient. This is important since, as noted above, nanoneedles 65 (which are at least about 5 mm long and less than about 60 microns in diameter, and preferably have an interior lumen of at least about 10 microns) tend to buckle easily, due to their extremely small size, their height-to-width aspect ratio, and the column strength attainable with current materials. See, for example, FIGS. 15-17, which show the tendency of (i) a “free” nanoneedle to buckle, (ii) a “pin-cuff” nanoneedle to buckle, and (iii) a “fixed cuff” nanoneedle to buckle.

It will be appreciated that, as a result of the foregoing construction, since spring tabs 95 bias movable guide plate 85 away from fixed guide plate 75 and spring tabs 100 bias movable guide plate 85 away from movable base plate 60, movable guide plate 85 moves in conjunction with movable base plate 60 and fixed guide plate 75 when movable base plate 60 is moved distally. Thus, movable guide plate 85 provides moving continuous lateral support to nanoneedles 65 during distal movement of nanoneedles 65 (i.e., as nanoneedles 65 are projected from the distal end of nanoneedle assembly 15 inserted into the skin of a patient).

With this form of the present invention, at the time of use, nanofluidic delivery system 5 has its peel-away strip 40 removed from the bottom surface of flexible member 20 of carrier 10, whereby to expose fixed guide plate 75 and gel reservoir 55. The bottom side of nanofluidic delivery system 5 is placed against the skin of a patient at the desired delivery site, and then dome 25 of carrier 10 is depressed, i.e., it is pushed toward the skin of the patient. Initial depressing of dome 25 of carrier 10 causes movable base plate 60 to advance distally within tubular body 50, whereby to advance nanoneedles 65 distally, out of fixed guide plate 75 and into the skin of the patient. More particularly, as dome 25 is depressed, the substance contained in reservoir 35 exerts a force on movable base plate 60, thereby moving movable base plate 60 distally. As this occurs, movable guide plate 85 also moves distally within tubular body 50, towards fixed guide plate 75, whereby to provide moving support for the advancing nanoneedles 65. In this way, nanoneedles 65 can be advanced through the skin of the patient without buckling. Further (and/or continued) depressing of dome 25 of carrier 10 causes the substance contained within reservoir 35 of dome 25 to pass into and through nanoneedles 65 and into the tissue of the patient. It will also be appreciated that the force used to move movable base plate 60 distally may be provided directly by the finger of the user as it depresses dome 25. In other words, the finger of the user may directly engage and move movable base plate 60.

### Nanoneedles

The nanoneedles 65 utilized in nanoneedle assembly 15 of nanofluidic delivery system 5 may be formed in any manner consistent with the present invention.

Three different approaches for forming nanoneedles 65 will now be described.

### Nanoneedles Formed by Carbon Nanostructures

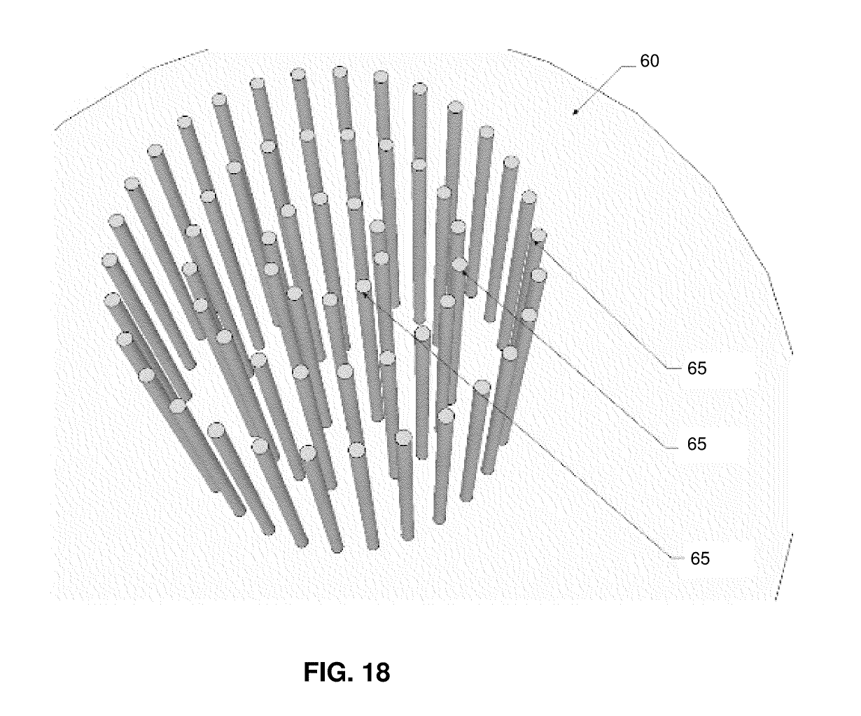

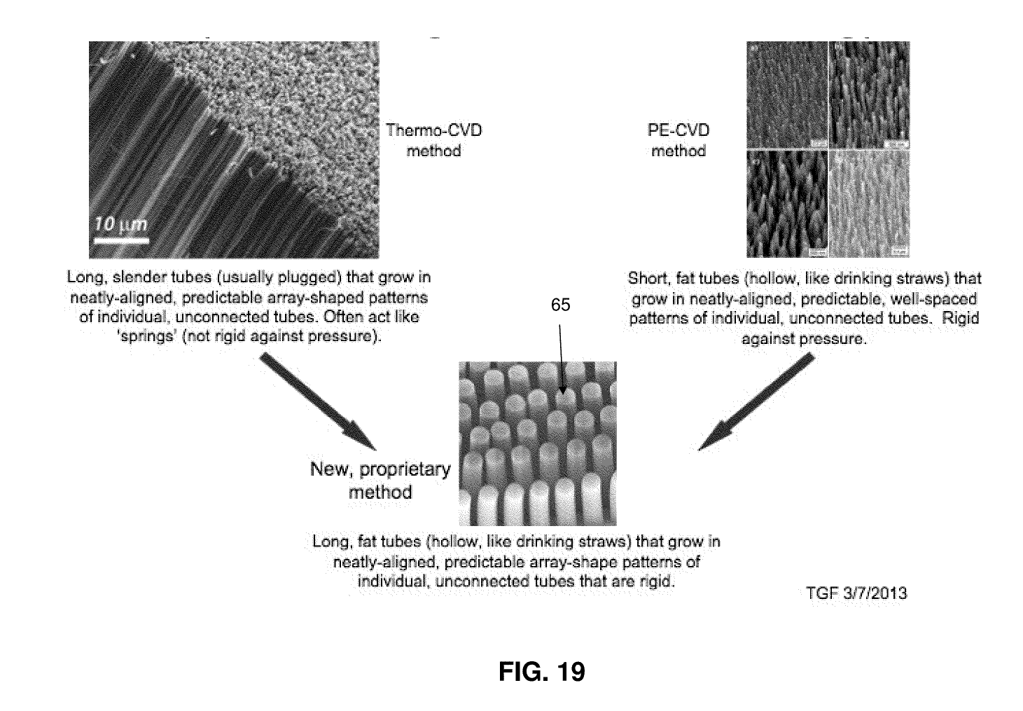

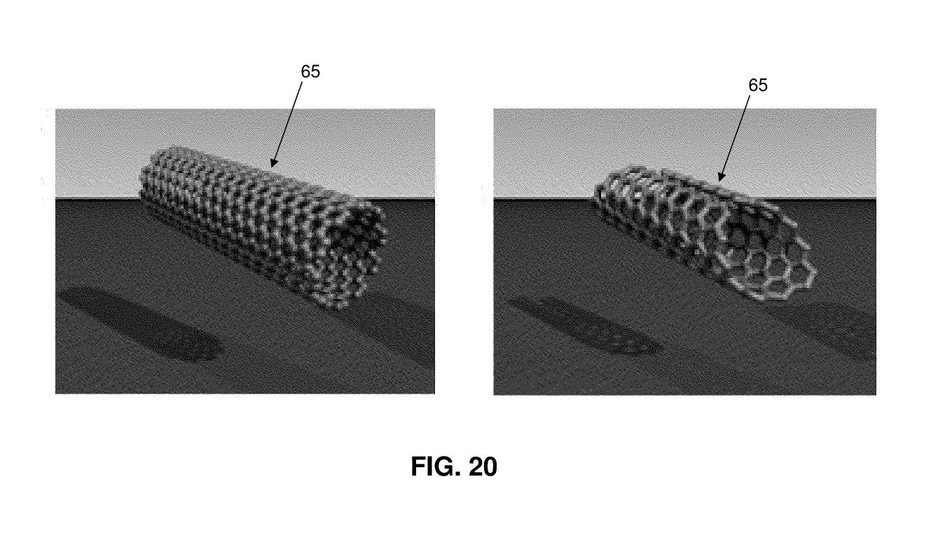

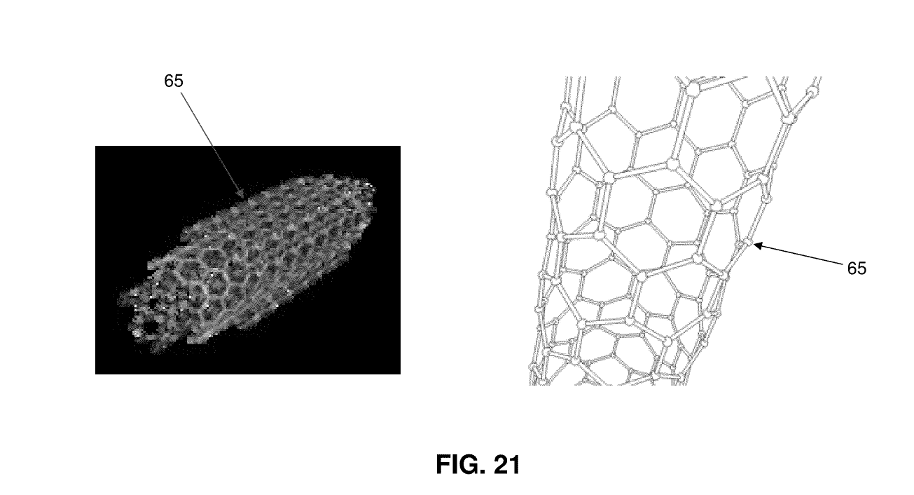

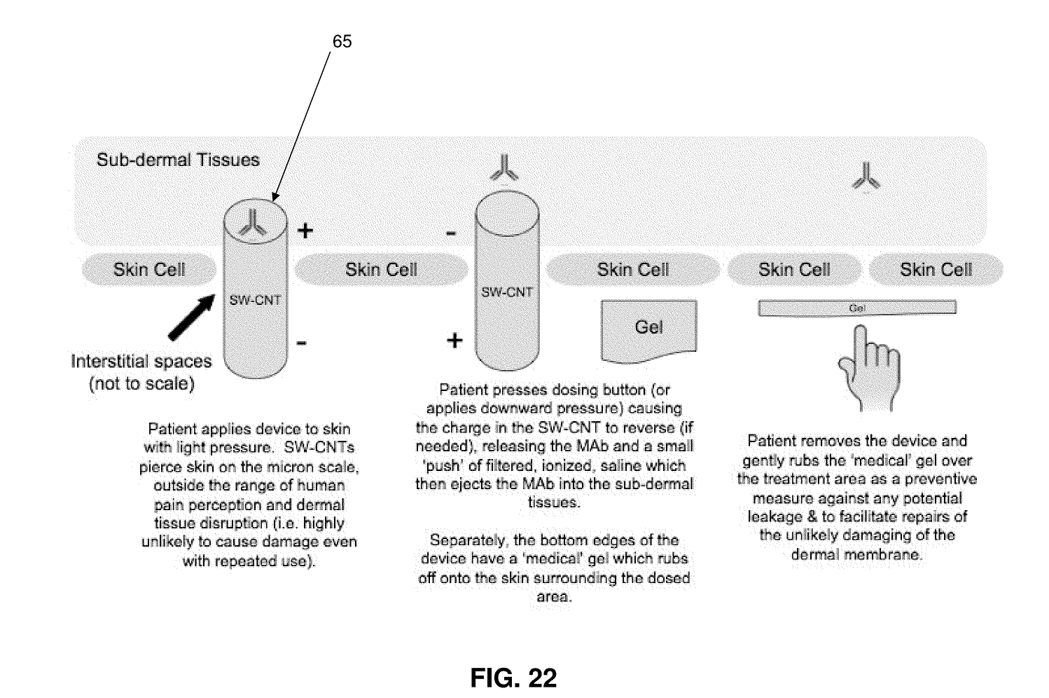

By way of example but not limitation, and looking now at FIGS. 18-22, each nanoneedle 65 may comprise a single carbon nanostructure such as a carbon nanofiber (CNF) or a carbon nanotube (CNT). These carbon nanotubes (CNTs) may be single-walled CNTs (FIG. 20) or multi-walled CNTs (FIG. 21). Such single-walled CNTs and multi-walled CNTs are well known in the art of carbon nanotubes.

### Nano-Needle Comprising a Plurality of Nanofibers (e.g., CNTs) Arranged to Form a Hollow Tubular Meta-Structure

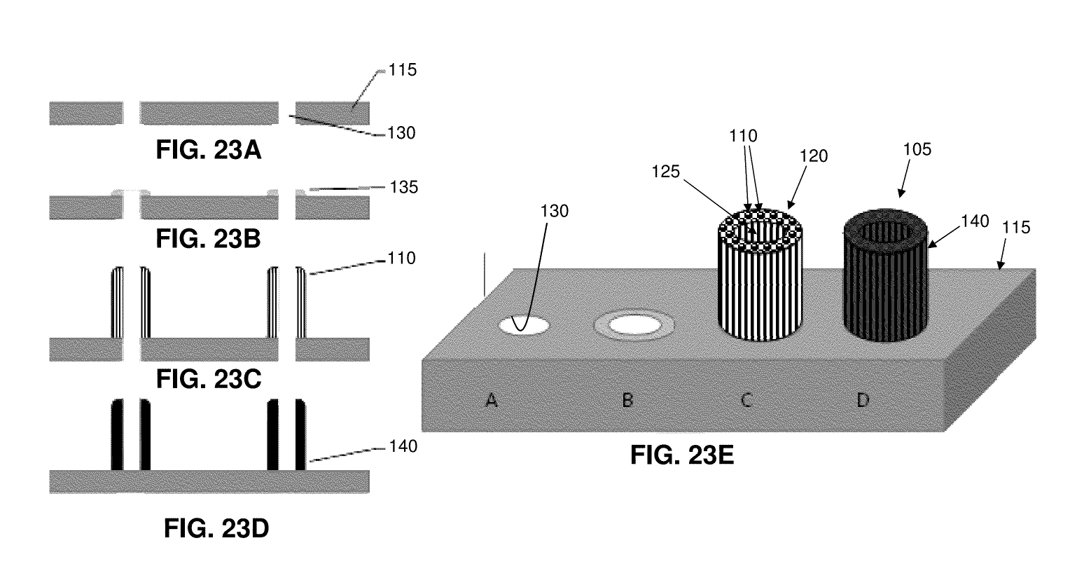

By way of further example but not limitation, and looking now at FIGS. 23A-23E, 24 and 25, each nanoneedle 65 may comprise a plurality of nanofibers (e.g., CNTs).

More particularly, and looking now at FIGS. 23A-23E, 24 and 25, there is provided a nanoneedle 105 comprising a plurality of nanofibers (e.g., CNTs) 110 extending out of a wafer substrate 115 and arranged so as to collectively form a hollow tubular meta-structure 120 having a lumen 125 defined thereby, with hollow tubular meta-structure 120 thereafter being sealed (as will hereinafter be discussed) so as to form nanoneedle 105 (which is analogous to the aforementioned nanoneedle 65). In this form of the invention, wafer substrate 115 comprises an opening 130 extending therethrough, so as to allow lumen 125 of nanoneedle 105 to communicate with the substance which is to be delivered, such that the substance which is to be delivered flows through lumen 125 of nanoneedle 105.

FIGS. 23A-23E show an approach for manufacturing nanoneedle 105.

FIG. 23A shows the wafer substrate 115 that is perforated by one or more openings 130.

FIG. 23B shows a ring of catalyst 135 deposited around the periphery of openings 130. Catalyst 135 (e.g., iron, cobalt, nickel and/or another metal well known in the art of growing carbon nanotubes) is typically deposited via sputtering or evaporation techniques, and patterned using optical or electron beam lithography techniques. Multi-layer catalysts or adhesion promoting layers can also be used in catalyst ring 135 without departing from the scope of the present invention. In one preferred form of the invention, aluminum oxide is deposited atop the wafer substrate 115, before the catalytic layer is deposited, so as to promote adhesion.

FIG. 23C shows an array of CNTs 110 having been grown from catalytic ring 135. During the heating process that precedes carbon nanotube growth, the catalyst metal film, which is typically thin (e.g., approximately 1 nm) will “break up” into nanoscale islands. Each island then nucleates the growth of a carbon nanotube. A carbon nanotube will grow in a random direction until it encounters another growing carbon nanotube, at which point the carbon nanotubes may either become entangled with one another, or adhere to one another, and then grow as a pair or as a group. This tends to promote vertical alignment in the array of carbon nanotubes. In this way, the hollow tubular meta-structure 120, having a lumen 125 defined thereby, is grown out of wafer substrate 115, wherein lumen 125 of hollow tubular meta-structure 120 is aligned with opening 130 extending through wafer substrate 115.

In FIG. 23D, a matrix material 140 is deposited within the interstitial spaces between CNTs 110 so as to form a rigid, non-porous hollow nanoneedle 105 having an inner and outer diameter that is roughly defined by catalyst ring 135, and a length that is defined by the height of the nanotube array, which is governed by process conditions and growth time. The deposition of a matrix material in the interstitial spaces between the nanotubes is discussed in Nicholas: “Electrical device fabrication from nanotube formations,” US 20100140591 A1. This filing discusses the use of chemical vapor deposition and atomic layer deposition to embed and encapsulate the nanotubes completely, and references Gordon et al., “ALD of High-k dielectrics on suspended functionalized SWNTs, Electrochemical and Solid-State Letters,” 8 (4) G89-G91 (2005) and Lu et al., “DNA Functionalization of Carbon Nanotubes for Ultra-Thin Atomic Layer Deposition of High k Dielectrics for nanotube Transistors with 60 mV/decade Switching,” arXiv:cond-mat/0602454; and Fahlman et al., “CVD of Conformal Alumina Thin Films via Hydrolysis of AlH₃(NMe₂Et),” Adv. Mater. Opt. Electron 10, 135-144 (2000).

See FIG. 23E, which provides an isometric, sequential view of the aforementioned four-step process for producing nanoneedle 105.

Note that in this form of the invention, the individual CNTs 110 may be substantially hollow, substantially solid or a combination thereof.

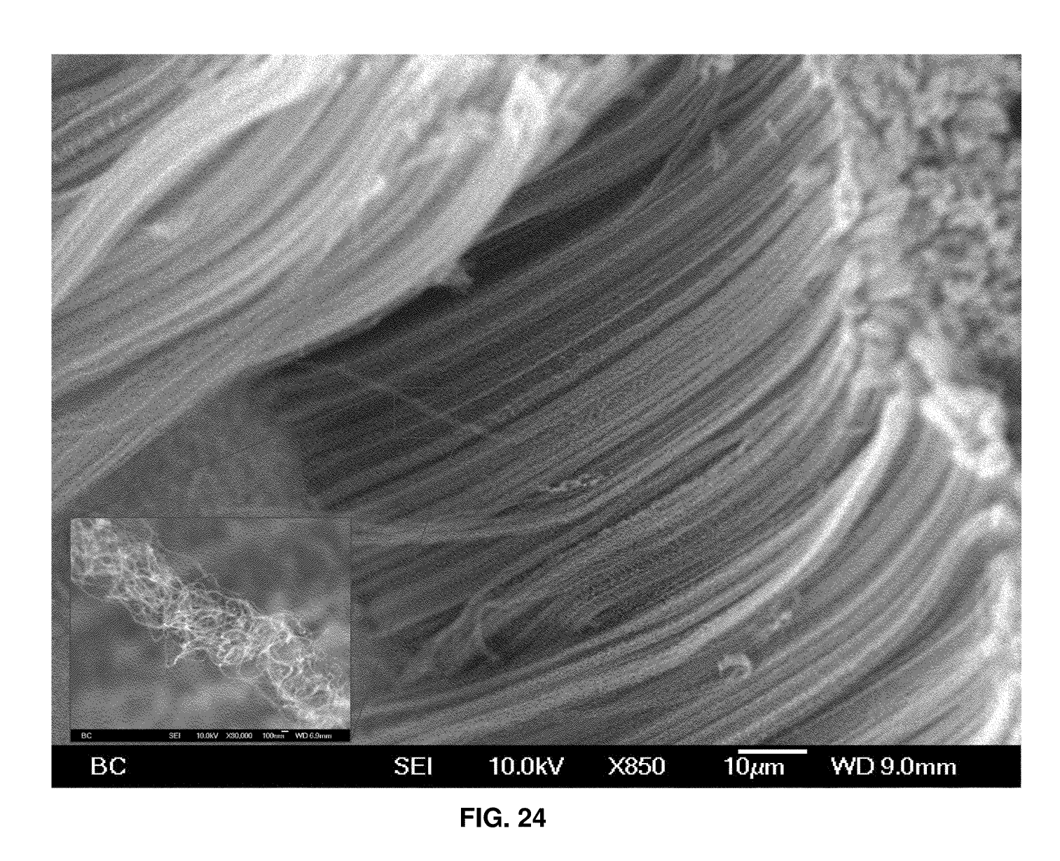

FIG. 24 shows an aligned array of CNTs 110 at low magnification. In the inset of FIG. 24, a cluster of CNTs 110 is shown, having overall parallel alignment despite significant directional wander of the constituent CNTs.

FIG. 25 shows nanoneedle 105 after a matrix material 140 has been deposited within the interstitial spaces between CNTs 110.

### Nanoneedles Formed by Sacrificial Fibers Overplated with a Rigid Material

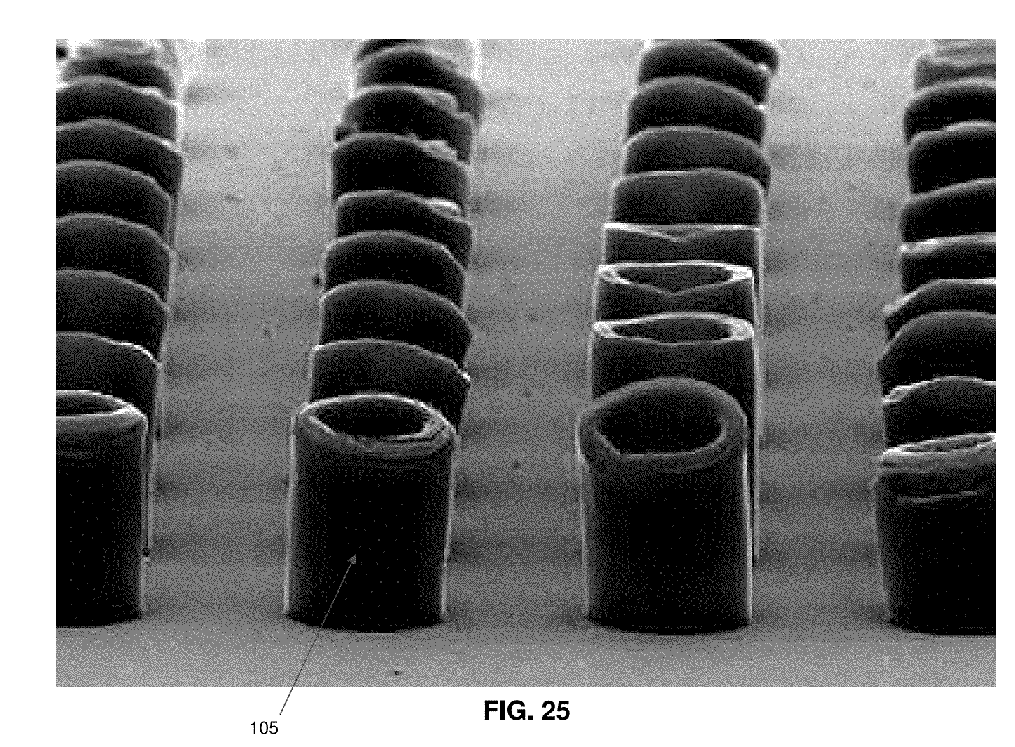

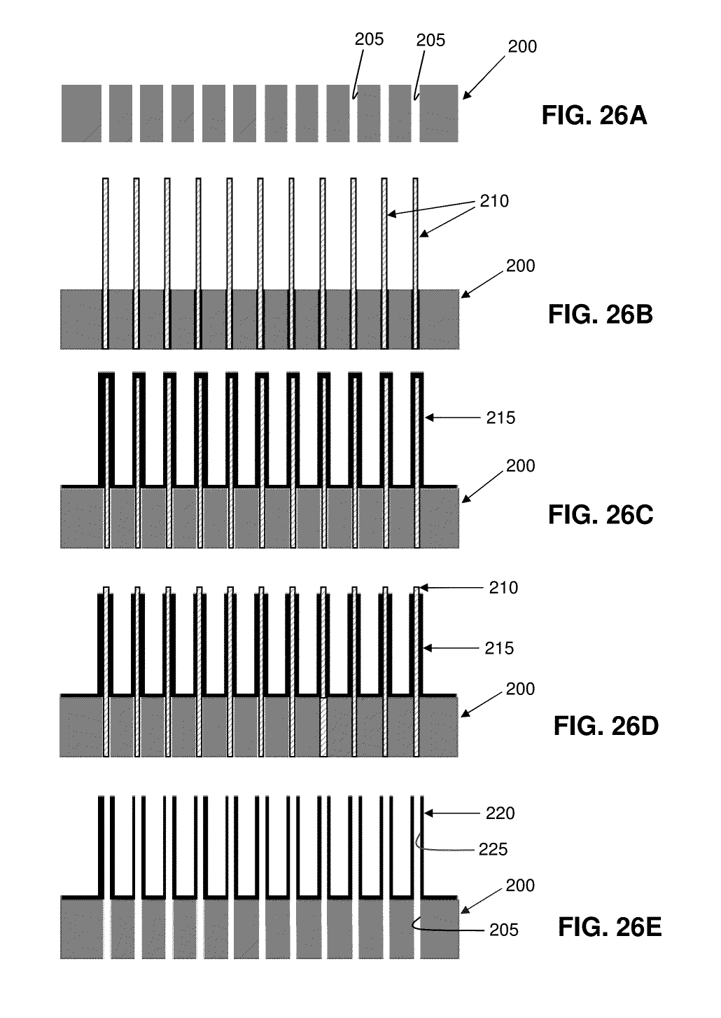

By way of further example but not limitation, nanoneedles 65 and/or nanoneedles 105 may be replaced by tubular structures formed using the process shown in FIGS. 26A-26E. More particularly, with this process, a support plate 200, having holes 205 extending therethrough, is provided (FIG. 26A). Solid fibers 210 are inserted into, and fixed to, support plate 200 such that each fiber is supported and freestanding, with spacing between adjacent fibers (FIG. 26B). Fibers 210 are then overcoated with a stiff material 215 (FIG. 26C). This fiber overcoating process may utilize any one of several common coating processes, including chemical vapor deposition, plating, physical vapor deposition (sputtering or evaporation), atomic layer deposition, spraying, dipping, electrophoretic deposition or the like. Fixation may include sintering, heat treating, solvent welding, etc. The stiff material 215 overcoating the free ends of fibers 210 is then removed, whereby to expose fibers 210 (FIG. 26D). Fibers 210 are then selectively etched away, without etching stiff material 215, whereby to leave hollow tubes 220 of stiff material 215 extending out of support plate 200, with the lumens 225 of hollow tubes 220 communicating with holes 205 in support plate 200 (FIG. 26E).

Various materials consistent with this approach may be used to form support plate 200, fibers 210, stiff material 215 and the preferential etchant. Of course, the selection of these materials must be coordinated with one another so as to be consistent with this fabrication process.

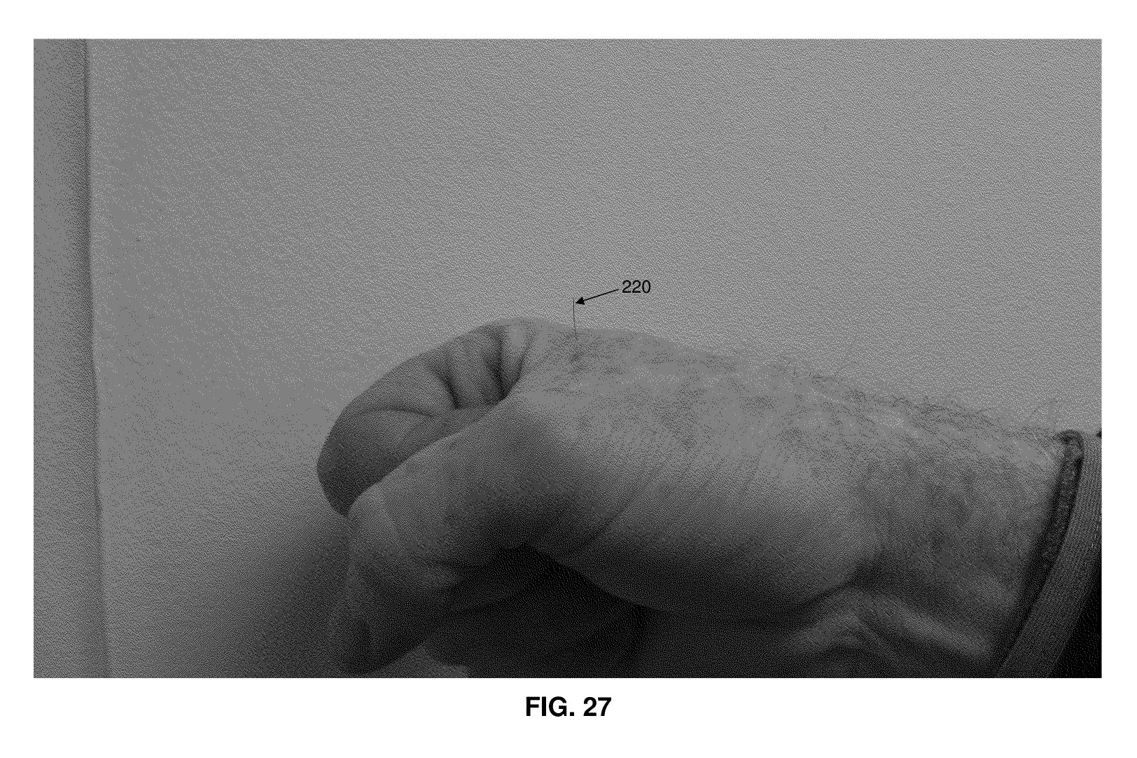

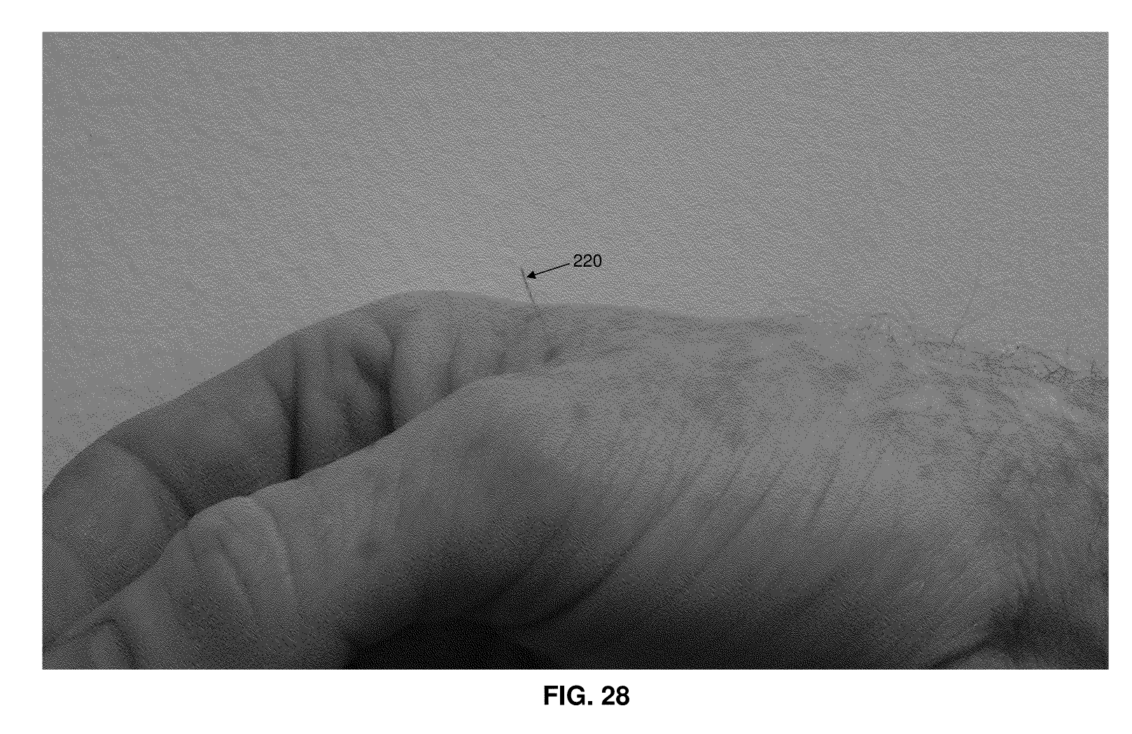

By way of example but not limitation, in one preferred form of the invention, stiff material 215 comprises tungsten, whereby to form tungsten hollow tubes 220. In this form of the invention, support plate 200 may comprise an etch-resistant material, fibers 210 may comprise plastics, glass, a ceramic, a low melting metal, or a readily etchable metal, and the preferential etchant may comprise hydrofluoric acid for the glass fibers, or a solvent for the plastic fibers. FIGS. 27 and 28 show an exemplary tungsten hollow tube 220, formed in accordance with the process depicted in FIGS. 26A-26E, extending out of the skin of a patient.

By way of further example but not limitation, in another preferred form of the invention, stiff material 215 comprises alumina, whereby to form alumina hollow tubes 220. In this form of the invention, support plate 200 may comprise either a plastic or a ceramic, fibers 210 may comprise plastic, glass or metals, and the preferential etchant may comprise solvents for plastic fibers, or HF for glass fibers, or HCl for ferrous metal fibers.

In general, it is preferred that support plate 200 comprises one from the group consisting of stainless steel or another metal, plastics or ceramics.

In general, it is preferred that fibers 210 comprise at least one from the group consisting of glass, carbon or a ceramic.

In general, it is preferred that stiff material 215 comprises at least one from the group consisting of a metal, ceramic or diamond-like carbon.

In general, it is preferred that the preferential etchant comprises at least one from the group consisting of 1:1 HF:HNO₃; 1:1 HF:HNO₃ (thin films); 3:7 HF:HNO₃; 4:1 HF:HNO₃ (rapid attack); 1:2 NH₄OH:H₂O₂ (thin films good for etching tungsten from stainless steel, glass, copper and ceramics, will also etch titanium as well); 305 g:44.5 g:1000 ml K₃Fe(CN)₆:NaOH:H₂O (rapid etch); HCl (slow etch, dilute or concentrated); HNO₃ (very slow etch, dilute or concentrated); H₂SO₄ (slow etch, dilute or concentrated); HF (slow etch, dilute or concentrated); H₂O₂; 1:1, 30%:70%, or 4:1 HF:HNO₃; 1:2 NH₄OH:H₂O₂; 4:4:3 HF:HNO₃:HAc; CBrF3 RIE etch; 305 g:44.5 g:1000 ml K₃Fe(CN)₆:NaOH:H₂O (very rapid etch); HCl solutions (slow attack); HNO₃ (slight attack) Aqua Regia 3:1 HCL:HNO₃ (slow attack when hot or warm); H₂SO₄ dilute and concentrated (slow etch); HF dilute and concentrated (slow etch); and Alkali with oxidizers (KNO3 and PbO2) (rapid etch).

### EXAMPLE 1

A roving of 15 micron diameter glass filament was debundled into individual filaments and processed in a chemical vapor deposition chamber. A tungsten coating, 20 microns thick, was deposited on the filaments, leading to the growth in the diameter of the filaments to 55 microns. The coated filaments were then cut to length, and immersed in an HF bath for several days. The disparity in the etch rates of tungsten and glass by hydrofluoric acid enables the glass core to be etched out, leaving the tungsten intact. However, the process is retarded by the limited area of glass exposed to the acid. Once etched, one end of each tungsten hollow needle was placed into holes in a Lexan support plate, so that each hollow needle was vertically oriented and freestanding. The solvent dicholoromethane was used to solvent-weld the tungsten tubes to the Lexan.

### EXAMPLE 2

As the individual handing required in Example 1 was arduous, a second process was developed to process the filaments in parallel. A length of 15 micron OD glass fiber roving was debundled and one end of each fiber was inserted into a stainless steel support plate, 0.1 mm thick, which had been laser drilled with 15 micron holes to receive the fibers. The plate thickness to hole diameter ratio in this case is approximately 6.6:1, which has been found sufficient to fixate the filaments, and within the capability of laser drilling. The glass fibers were then overcoated with tungsten by a CVD process, which also covered the stainless support plate, all to a thickness of 20 microns. The backside was protected to prevent coating on the backside of the support plate. The tungsten coating at the fiber tips was exposed to an etchant, (K₃Fe(CN)₆:NaOH:H₂O 30.5 g:4.45 g:100 ml) to re-expose the glass fibers. The glass fibers were then etched out with hydrofluoric acid, leaving an array of hollow needles, vertically standing where their glass fiber cores had once been. The process followed in this example is illustrated in FIGS. 26A-26E.

### EXAMPLE 3

Lengths of 15 micron palladium wire were passed through a copper coated polyimide support sheet, such that each wire protruded from the support plate by 5 mm on the metallized side, and protruded by a smaller amount on the side without the metallization. The palladium wires and copper surface were dipped into an alumina ceramic slurry and a DC voltage was applied to cause electrophoretic deposition on the copper and wires, which served as the cathode. The polyimide support was then removed, leaving a ceramic deposit both where the metallized polyimide had been, and also around the wires. The wires were carefully removed, and the ceramic article sintered to create a plate with hollow needles. The needles were not universally open after this process, so the article was potted in a wax, then polished on a silicon carbide paper to expose the inner diameter. The wax was then removed, leaving the article with the holes exposed.

### Modifications

While the present invention has been described in terms of certain exemplary preferred embodiments, it will be readily understood and appreciated by those skilled in the art that it is not so limited, and that many additions, deletions and modifications may be made to the preferred embodiments discussed herein without departing from the scope of the invention.

What is claimed is:

1. Apparatus for subcutaneously delivering a substance to a patient, said apparatus comprising:
a carrier comprising a flexible body, wherein said flexible body comprises a reservoir, and further wherein said reservoir contains the substance which is to be delivered to the patient;
a nanoneedle assembly comprising:
a tubular body having a distal end and a proximal end;
a base plate movably mounted intermediate said distal end and said proximal end of said tubular body, said base plate comprising a distal surface and a proximal surface, with a plurality of through-holes extending between said distal surface and said proximal surface of said base plate, said proximal surface of said base plate being in fluid communication with said reservoir;
a plurality of nanoneedles, wherein each of said plurality of nanoneedles comprises a distal end, a proximal end, and a lumen extending therebetween, said proximal end of each of said plurality of nanoneedles being mounted to said base plate such that said lumen of each of said plurality of nanoneedles is in fluid communication with said through-holes of said base plate;
a fixed guide plate mounted at said distal end of said tubular body, said fixed guide plate comprising a plurality of through-holes extending therethrough, said through-holes of said fixed guide plate being sized to receive said distal ends of said plurality of nanoneedles; and
a moveable guide plate disposed intermediate said base plate and said fixed guide plate, said moveable guide plate comprising a plurality of through-holes extending therethrough, said through-holes of said movable guide plate being sized to receive said plurality of nanoneedles, such that said plurality of nanoneedles extend through said through-holes of said movable guide plate; and
at least one spring tab for biasing said movable guide plate away from said fixed guide plate;
wherein, when said base plate is moved distally, said movable guide plate moves distally, such that said movable guide plate provides lateral support to said nanoneedles, whereby to prevent buckling of said nanoneedles; and
wherein when said base plate moves distally, said distal end of each of said plurality of nanoneedles passes through said through-holes of said fixed guide plate into the patient, and further wherein when said distal ends of said plurality of nanoneedles are disposed distally of said fixed guide plate, the substance within said reservoir passes through each of said lumens of said plurality of nanoneedles, whereby to deliver the substance to the patient.

2. Apparatus according to claim 1, wherein said nanoneedle assembly further comprises at least one spring tab for biasing said movable guide plate away from said base plate.

3. Apparatus according to claim 1 wherein said nanoneedle assembly further comprises a gel reservoir configured to release gel at the distal end of the tubular body.

4. Apparatus according to claim 3 wherein said gel reservoir is disposed circumferentially around the distal end of said tubular body.

5. Apparatus according to claim 3 wherein said gel reservoir further comprises a plurality of air vents for facilitating the release of gel from said gel reservoir.

6. Apparatus according to claim 1 wherein each of said plurality of nanoneedles is long enough to penetrate the skin of the patient and narrow enough to avoid causing pain to the patient.

7. Apparatus according to claim 6 wherein each of said plurality of nanoneedles is at least about 5 mm in length, less than 50 microns in diameter and has an interior lumen of at least about 10 microns in diameter.

8. Apparatus according to claim 1 wherein a sufficient number of nanoneedles are provided so as to deliver the desired quantity of the substance from said reservoir to the patient within a desired time.

9. Apparatus according to claim 1 wherein each of said plurality of nanoneedles is formed of a single carbon nanostructure.

10. Apparatus according to claim 1 wherein each of said plurality of nanoneedles comprises a plurality of nanofibers disposed around the periphery of said through-holes in said base plate, wherein the interstitial spaces between said nanofibers are filled by a matrix material.

11. Apparatus according to claim 1, wherein each of said plurality of nanoneedles comprises a tubular structure.

12. Apparatus according to claim 1 wherein said carrier further comprises a peel-away strip extending across the distal end of said flexible body so as to seal said nanoneedle assembly within said carrier.

13. Apparatus according to claim 12, wherein said peel-away strip comprises a pull tab to facilitate the removal of said peel-away strip from said carrier.

14. A method for subcutaneously delivering a substance to a patient, said method comprising:
providing apparatus comprising:
a carrier comprising a flexible body, wherein said flexible body comprises a reservoir, and further wherein said reservoir contains the substance which is to be delivered to the patient;
a nanoneedle assembly comprising:
a tubular body having a distal end and a proximal end;
a base plate movably mounted intermediate said distal end and said proximal end of said tubular body, said base plate comprising a distal surface and a proximal surface, with a plurality of through-holes extending between said distal surface and said proximal surface of said base plate, said proximal surface of said base plate being in fluid communication with said reservoir;
a plurality of nanoneedles, wherein each of said plurality of nanoneedles comprises a distal end, a proximal end, and a lumen extending therebetween, said proximal end of each of said plurality of nanoneedles being mounted to said base plate such that said lumen of each of said plurality of nanoneedles is in fluid communication with said through-holes of said base plate;
a fixed guide plate mounted at said distal end of said tubular body, said fixed guide plate comprising a plurality of through-holes extending therethrough, said through-holes of said fixed guide plate being sized to receive said distal ends of said plurality of nanoneedles; and
a moveable guide plate disposed intermediate said base plate and said fixed guide plate, said moveable guide plate comprising a plurality of through-holes extending therethrough, said through-holes of said movable guide plate being sized to receive said plurality of nanoneedles, such that said plurality of nanoneedles extend through said through-holes of said movable guide plate; and
at least one spring tab for biasing said movable guide plate away from said fixed guide plate;
wherein, when said base plate is moved distally, said movable guide plate moves distally, such that said movable guide plate provides lateral support to said nanoneedles, whereby to prevent buckling of said nanoneedles; and
wherein when said base plate moves distally, said distal end of each of said plurality of nanoneedles passes through said through-holes of said fixed guide plate into the patient, and further wherein when said distal ends of said plurality of nanoneedles are disposed distally of said fixed guide plate, the substance within said reservoir passes through each of said lumens of said plurality of nanoneedles, whereby to deliver the substance to the patient;
positioning said apparatus such that said distal end of said tubular body is disposed against the skin of the patient;
moving said base plate distally so as to advance said plurality of nanoneedles into the skin of the patient; and
delivering the substance through said nanoneedles into the patient.

15. A method according to claim 14 wherein said base plate is moved distally by depressing said flexible body.

16. A method for forming a hollow tube, said method comprising:
providing a support plate having a plurality of holes extending therethrough;
inserting a plurality of fibers into said plurality of holes so as to mount said fibers to said support plate;
overcoating said fibers with a stiff material;
removing said stiff material from the ends of said fibers opposite said support plate, whereby to expose said fibers; and
selectively etching away said fibers so as to leave hollow tubes of said stiff material extending from said support plate.

17. A method according to claim 16 wherein said fibers are selected from the group consisting of plastics, glass, a ceramic, a low melting metal or a readily etchable metal.

18. A method according to claim 16 wherein said stiff material is formed by one from the group consisting of chemical vapor deposition, plating, physical vapor deposition, atomic layer deposition, spraying, dipping and electrophoretic deposition.

19. A method according to claim 16 wherein said stiff material comprises one from the group consisting of tungsten and alumina.

20. A method according to claim 16 wherein said support plate comprises one from the group consisting of stainless steel, plastics, ceramics and metal.
